(19) United States
(12) Patent Application Publication
(10) Pub. No.: US 2011/0053879 A1
(43) Pub. Date: Mar. 3, 2011

(54) PICOPLATIN AND AMRUBICIN TO TREAT LUNG CANCER

(75) Inventors: Ronald A. Martell, San Francisco, CA (US); David A. Karlin, Los Altos, CA (US)

(73) Assignee: Poniard Pharmaceuticals, Inc., Seattle, WA (US)

(21) Appl. No.: 12/866,710

(22) PCT Filed: Feb. 6, 2009
(86) PCT No.: PCT/US09/00750

§ 371 Date: Nov. 10, 2010

Related U.S. Application Data
(60) Provisional application No. 61/027,360, filed on Feb. 8, 2008.
(60) Provisional application No. 61/027,382, filed on Feb. 8, 2008.
(60) Provisional application No. 61/027,387, filed on Feb. 8, 2008.

(51) Int. Cl. A61K 31/704; A61P 35/04

(52) U.S. Cl. 514/34

(57) ABSTRACT

A method for treatment of lung cancer comprising administration of picoplatin and amrubicin, or comprising radiation therapy and picoplatin is provided. A use of picoplatin in conjunction with amrubicin for treatment of lung cancer is provided. The lung cancer can be SCLC or NSCLC. The cancer can be resistant or refractory to treatment or that progresses following cessation of first-line organoplatinum chemotherapy. The treatment can include the administration of picoplatin and amrubicin, optionally in conjunction with a regimen of best supportive care. Multiple doses of the drug or drug combination can be administered.

### CROSS-REFERENCE TO RELATED APPLICATIONS

This application claims the priority of U.S. Ser. No. 61/027,387, filed Feb. 8, 2008, U.S. Ser. No. 61/027,382, filed Feb. 8, 2008, and U.S. Ser. No. 61/027,360, filed Feb. 8, 2008, the disclosures of which are incorporated by reference herein in their entireties. This application also claims the priority of U.S. Ser. Nos. 60/857,066 (filed Nov. 6, 2006), 60/857,725 (filed Nov. 8, 2006), 60/877,495 (filed Dec. 28, 2006), 60/889,191 (filed Feb. 9, 2007), 60/931,589 (filed May 24, 2007), and 60/983,852 (filed Oct. 30, 2007), and of U.S. Ser. No. 11/982,841, filed Nov. 5, 2007, the disclosures of which are incorporated by reference herein in their entireties.

### BACKGROUND

Small cell lung cancer (SCLC) accounts for approximately 14% of all lung cancers. In 2004, there were approximately 26,000 new cases in the United States and 51,000 new cases in Europe (Jemal, 2004). The median survival of patients with untreated SCLC is two to four months (Clark, 1998; Glisson, 2003; Davies, 2004). Combination chemotherapy is currently considered standard first-line therapy for SCLC. The most common regimens include cisplatin or carboplatin and etoposide. Unfortunately, despite the 40-90% response rate to first-line chemotherapy, long-term survival is unusual because patients develop resistance to chemotherapy and relapse (Sundstrom, 2005; Jackman, 2005). The overall expected mean survival after disease relapse is two to four months (Huisman, 1999).

At the time of diagnosis, approximately 30% of patients with SCLC will have tumors confined to the ipsilateral chest, mediastinum, and supraclavicular nodes, designated limited disease. Initially 70-90% of these patients will respond to chemotherapy but the recurrence rate is high (75-90%). The median survival time of patients with limited disease ranges from 14 to 20 months with a two-year survival rate of 40%. Even with the addition of radiation therapy to the chest and head, only 6-15% of patients live beyond five years. Patients with more widespread, extensive disease, have an even worse prognosis. Although response rates to initial chemotherapy remain relatively high, i.e., 40-70%, the median survival of 9-11 months is shorter than for patients with limited disease and long-term survival is rare. Fewer than 5% of patients with extensive disease live beyond two years, even with multi-agent, intensive therapy.

Non-small cell lung cancer (NSCLC) is a heterogeneous aggregate of histologies. The most common histologies are epidermoid or squamous carcinoma, adenocarcinoma, and large cell carcinoma. These histologies are often classified together because approaches to diagnosis, staging, prognosis, and treatment are similar. Patients with resectable disease may be cured by surgery or surgery with adjuvant chemotherapy. Local control can be achieved with radiation therapy in a large number of patients with unresectable disease, but cure is seen only in a small number of patients. Patients with locally advanced, unresectable disease may have long-term survival with radiation therapy combined with chemotherapy. Patients with advanced metastatic disease may achieve improved survival and palliation of symptoms with chemotherapy.

At diagnosis, patients with NSCLC can be divided into three groups that reflect both the extent of the disease and the treatment approach. The first group of patients has tumors that are surgically resectable. Patients with resectable disease who have medical contraindications to surgery are candidates for curative radiation therapy. The second group includes patients with either locally (T3-T4) and/or regionally (N2-N3) advanced lung cancer. The final group includes patients with distant metastases (M1) that were found at the time of diagnosis. This group can be treated with radiation therapy or chemotherapy for palliation of symptoms from the primary tumor. Platinum-based chemotherapy has been associated with short-term palliation of symptoms and with a survival advantage. Currently, no single chemotherapy regimen can be recommended for routine use.

Cisplatin, cis-dichlorodiammine platinum (II), the first organoplatinum anticancer drug, was introduced approximately 30 years ago and is still widely used in the treatment of various solid tumors in human patients, and possesses a wide range of activity against different tumor types. However, cisplatin also exhibits a number of undesirable side effects, such kidney damage (nephrotoxicity) and nausea and vomiting. The search for organoplatinum compounds with fewer side effects than cisplatin led to the discovery of carboplatin (cis-diammine-1,1-cyclobutane dicarboxylate platinum (II)), but this compound also exhibits nephrotoxicity and myelotoxicity and is known to cause cumulative dose-related toxicity that results in slow bone marrow recovery. More recently oxaliplatin (trans-1,2-cyclohexane-diammine oxalate platinum (II)) was also developed, but this compound possesses significant neurotoxicity, although its nephrotoxicity was reduced relative to carboplatin. Other platinum-containing drugs that are being studied include satraplatin and lobaplatin. In addition to their undesirable side effects, these organoplatinum compounds are not effective against all tumor types and, significantly, tumors can mutate to develop resistance or tolerance to these compounds, resulting in a tumor that can no longer be controlled with these compounds.

There is currently no second-line therapy approved by the United States Food and Drug Administration (FDA) for treatment of patients with refractory or resistant SCLC. These patients have an extremely poor prognosis. The response rate is <10% for any single-agent regimen in this group of patients (Davies, 2004; Murray, 2003; Sundstrom, 2005; NCCN, 2008) The National Comprehensive Cancer Network (NCCN) 2008 guidelines indicate that monotherapy with ifosfamide, paclitaxel, docetaxel, gemcitabine, or topotecan may be used. These agents, however, have shown neither a significant response rate nor survival benefit and their use in this population is often associated with drug-related toxicities. There is a high degree of consensus in the published literature that no currently available therapy offers significant benefit to patients who have refractory or resistant disease.

Thus, there clearly remains an unmet need for improved chemotherapy for lung cancer. Also needed are combination therapies that can provide improved treatment for refractory, resistant, and 91-180 day progressive lung cancer.

### SUMMARY

The present invention is directed to methods of treatment of lung cancer patients comprising the use of picoplatin and amrubicin; to uses of picoplatin and amrubicin in the treatment of lung cancer, and to pharmaceutical compositions comprising picoplatin and amrubicin.

In various embodiments, the invention provides a method for treating lung cancer in a human comprising administering to a human afflicted with lung cancer an effective anti-cancer amount of picoplatin and an effective anti-cancer amount of amrubicin.

In various embodiments, the invention provides the use of an effective anti-cancer amount of picoplatin in conjunction with an effective anti-cancer amount of amrubicin for treating lung cancer in a human afflicted with lung cancer.

In various embodiments, the invention provides a method for treating lung cancer comprising:

(a) selecting a population of human patients for treatment with picoplatin and amrubicin, wherein said patients are afflicted with lung cancer that was refractory to initial treatment or that responded to initial treatment and wherein the lung cancer then progressed within 180 days from the last day of the initial treatment,

(b) selecting a subpopulation of patients from said population for treatment with picoplatin and amrubicin wherein said subpopulation consists of patients whose lung cancer progressed within 91-180 days from the last day of the initial treatment;

(c) administering picoplatin and amrubicin to said patients selected for treatment; and

(d) optionally, concomitantly with step (c), providing to the patients a regimen of best supportive care (BSC),

so that the life of the patient is extended over that of a patient not receiving step (c).

In various embodiments, the invention provides a pharmaceutical composition comprising picoplatin and amrubicin, and a pharmaceutically acceptable aqueous carrier, formulated for intravenous administration to a human.

In various embodiments, the invention provides a method for treating lung cancer comprising treating a human patient afflicted with lung cancer with radiation therapy followed by administering to the patient an effective anti-cancer amount of picoplatin and, optionally, amrubicin.

### DETAILED DESCRIPTION

References in the specification to “one embodiment” or “an embodiment” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. The term “various embodiments” refers to one or a plurality of embodiments, but not necessarily to all embodiments, according to the present invention.

Unless otherwise indicated, the words and phrases presented in this document have their ordinary meanings to one of skill in the art. Such ordinary meanings can be obtained by reference to their use in the art and by reference to general and scientific dictionaries, for example, Webster's Third New International Dictionary, Merriam-Webster Inc., Springfield, Mass., 1993, The American Heritage Dictionary of the English Language, Houghton Mifflin, Boston Mass., 1981, and Hawley's Condensed Chemical Dictionary, 14^{th} edition, Wiley Europe, 2002.

The term “treatment” is defined as the management and care of a patient for the purpose of combating the disease, condition, or disorder and includes administering a compound of the present invention to prevent the onset of the symptoms or complications, or alleviating the symptoms or complications, or eliminating the disease, condition, or disorder.

“Treating” within the context of the instant invention means an alleviation of symptoms associated with a disorder or disease, or inhibition of further progression or worsening of those symptoms, or prevention or prophylaxis of the disease or disorder. Thus, treating a cancer or metastatic disease includes slowing, halting or reversing the growth of the disease and/or the control, alleviation or prevention of symptoms of the disease. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result by inhibition of malignant growth activity or metastatis. A therapeutically effective amount is also one in which any toxic or detrimental effects of compounds of the invention are outweighed by the therapeutically beneficial effects. For example, in the context of treating cancer, a therapeutically effective amount of a picoplatin or amrubicin refers to an amount sufficient to have a beneficial effect in treatment of the cancer, such as lung cancer.

In various embodiments, the invention provides a method for treating lung cancer in a human comprising administering to a human afflicted with lung cancer an effective anti-cancer amount of picoplatin and an effective anti-cancer amount of amrubicin. The lung cancer can be small cell lung cancer (SCLC). Alternatively, the lung cancer can be non-small cell lung cancer (NSCLC).

The picoplatin and the amrubicin can be administered in one or more doses, and optionally concurrently providing to the patient a regimen of best supportive care (BSC). The administration of the picoplatin can be oral, intravenous, or a combination thereof, and the administration of the amrubicin can be oral, intravenous, or a combination thereof. In one embodiment, the administration of the picoplatin is oral.

The picoplatin can be administered once a day on day one of a two to four week treatment cycle, wherein at least two cycles of treatment are carried out. A dose of about 5 mg/m² to about 150 mg/m² of picoplatin can be administered. The amrubicin can be administered once a day for one to three days starting on day one of a two to four week treatment cycle, and at least two cycles of treatment can be carried out. A daily dose of about 5 mg/m² to about 45 mg/m² of amrubicin can be administered. Additionally, the picoplatin, the amrubicin, or both, can be administered in an initial treatment dose (or doses for amrubicin), and then administered again at about seven day intervals thereafter.

In one embodiment, the treatment cycle is a 21 day treatment cycle. The treatment cycle can be increased or decreased, for example by one or two weeks, depending on patient response to the treatment. In one specific embodiment, the picoplatin is administered daily for one day starting on day one of a 21 day treatment cycle and the amrubicin is administered daily for the first three days of the 21 day treatment cycle.

The picoplatin and amrubicin combination therapy can be a first-line therapy wherein the lung cancer has not been previously treated with radiation or with any other chemotherapeutic agents. Alternatively, the therapy can be used when a patient is refractory, resistant, or relapsed/progressive within 91-180 days, after cessation of first-line chemotherapy and/or radiation treatment.

The treatment can be used as a first-line therapy for SCLC with extensive disease. The treatment can also be a first-line therapy for SCLC with limited disease wherein the treatment is administered in conjunction with radiation therapy. Alternatively, the treatment can be a second-line therapy for SCLC with extensive or limited disease that is refractory to initial chemotherapy or progressive within 6 months of completing first line, platinum-containing therapy.

Also, the treatment can be a first-line therapy for NSCLC with extensive disease. The treatment can be a first-line therapy for NSCLC with limited disease and the treatment is administered in conjunction with radiation therapy. Alternatively, the treatment can be a second-line therapy for NSCLC with extensive or limited disease that is refractory to initial chemotherapy or progressive within 6 months of completing first line, platinum containing therapy.

In various embodiments, the patient is first treated with radiation therapy, and/or treated with radiation therapy in conjunction with the treatments using picoplatin, and optionally amrubicin. For example, a patient can be treated with radiation therapy to sensitize the cancer for more effective treatment with picoplatin, or the picoplatin and amrubicin combination.

Various embodiments of the invention further provide a method for treating lung cancer comprising treating a human patient afflicted with lung cancer with radiation therapy followed by administering to the patient an effective anti-cancer amount of picoplatin, and optionally, amrubicin.

In various embodiments, the invention provides the use of an effective anti-cancer amount of picoplatin in conjunction with an effective anti-cancer amount of amrubicin for treating lung cancer in a human afflicted with lung cancer.

Furthermore, the present invention provides a method for treating lung cancer in a human comprising: administering to a human patient afflicted with lung cancer, that is refractory, resistant or relapsed/progressive within 91-180 days, after cessation (i.e., after the last dose) of first-line chemotherapy, picoplatin, which can be administered in at least two doses spaced at about three- to six-week intervals, and optionally concurrently providing to the patient a regimen of best supportive care (BSC). The treatment can additionally include administration of amrubicin, which can be administered in three daily doses on three consecutive days, at least twice, spaced at about three- to six-week intervals.

Patients with lung cancer who fail to respond or progress through first-line platinum containing chemotherapy with other platinum containing (Pt) agents are considered to be “refractory.” Patients who initially respond to initial or “first-line” chemotherapy comprising other platinum agents and then relapse/progress (PD) within 90 days (3 months) are considered to be “resistant.” Patients who respond to initial treatment but then relapse or whose tumors progress within about 91-180 days (—3-6 months) after the cessation of first-line therapy with other platinum agents are considered herein to have a “91-180 day progressive” lung cancer. The present method can result in control of the lung cancer and can extend the life of these patients. “Control” is defined as response (complete or partial, “PR”) or stable disease, i.e., absence of progression. The lung cancer can be small cell lung cancer (SCLC) or non-small cell lung cancer (NSCLC).

In various embodiments of the present method, the patient has not previously been treated for metastatic disease, or the patient has not previously had systemic treatment, such as chemotherapy, for localized or metastatic disease. For example, the patient may have had surgery to remove or to de-bulk the primary tumor and then be treated with one of the picoplatin, 5-FU, leucovorin regimens (e.g., FOLPI) of the invention to prevent or delay progression of the cancer, including to prevent or delay the development of metastases. The patient may have received earlier chemotherapy at the time of primary tumor treatment, at least 6 months prior to the present picoplatin treatment.

In various embodiments, the picoplatin can be administered with curative intent, rather than merely seeking to arrest the disease with no remission. The dosage of the picoplatin can be increased beyond that bringing about disease stasis in order to achieve a cure in the patient.

An embodiment of the present invention thus provides a method for treating lung cancer comprising: (a) selecting a population of human patients for treatment with picoplatin and amrubicin, wherein said patients are afflicted with lung cancer that was refractory to initial treatment or that responded to initial treatment and wherein the lung cancer then progressed within 180 days from the last day of the initial treatment, (b) selecting a subpopulation of patients from said population for treatment with picoplatin wherein said subpopulation consists of patients whose lung cancer progressed within 91-180 days from the last day of the initial treatment; (c) administering picoplatin and amrubicin to said patients selected for treatment; and (d) optionally, concomitantly with step (c), providing to the patients a regimen of best supportive care (BSC), so that the life of the patient is extended over that of a patient not receiving step (c).

In an embodiment of a method according to the invention, the picoplatin can be the only chemotherapeutic anti-cancer agent administered to the patient selected for treatment when the treatment is combined with radiation therapy, either prior to administration of picoplatin, and/or concurrently with the picoplatin administration. In another embodiment, picoplatin is administered to said patient in conjunction with an effective amount of at least one non-platinum anticancer agent.

The picoplatin can also be administered in conjunction with a concurrent treatment of BSC for SCLC and/or NSCLC as defined herein. Preferably, the present method extends the life of the patient and can also result in control of the lung cancer.

Patients whose lung cancer progresses about 91-180 days (3-6 months) after first-line chemotherapy have heretofore typically been treated as having sensitive tumors, but the inventors herein have recognized that such tumors that generally do not respond to, and therefore should not be retreated with, the first-line therapy, e.g., comprising organoplatinum compounds such as cisplatin or carboplatin, but rather be treated with organoplatinum compounds suitable for tumors that have developed resistance to such first-line organoplatinum compounds. The inventors herein have recognized that this population of patients with lung cancer that progresses within the 91-180 day period after cessation of the first-line therapy, as well as patients whose lung cancer is refractory to treatment and progresses within 180 days, or whose lung cancer responds to initial treatment and then progresses within 180 days of cessation of initial treatment (collectively referred to as “progressive within 180 days”), can advantageously be treated with picoplatin and amrubicin so as to increase their overall survival (lifespan), irrespective of any objective tumor response during treatment.

In one embodiment, the patients are selected from those afflicted with lung cancer that is progressive following initial treatment of the patient (“first-line therapy”) with another platinum-containing drug, such as cisplatin or carboplatin, in that the cancer responds to initial treatment, then progresses within 180 days, including those who respond to initial treatment and progress within about 91-180 days after cessation of the first-line treatment. In another embodiment, the patients are selected from those afflicted with lung cancer that is refractory to the initial previous treatment of the patient (“first-line therapy”) with another platinum-containing drug, such as cisplatin or carboplatin.

In one embodiment of the invention, about 60 mg/m²-150 mg/m², or in a second embodiment, preferably about 150 mg/m² of picoplatin is administered in each dose. Additionally, about 5 mg/m² to about 45 mg/m², or in a another embodiment, about 10, 15, 20, 25, 35, 40, or 45 mg/m² of amrubicin is administered in each dose. The doses may be administered orally or parenterally, or via combination of oral and parenteral routes. In one embodiment, the picoplatin doses are administered by intravenous infusion of an aqueous solution of picoplatin. The infusion of one dose is typically carried out over about one to two hours. The amrubicin doses can be administered by intravenous infusion of an aqueous solution of amrubicin. The infusion of one dose is typically carried out over about five minutes to about two hours.

The solution containing picoplatin and amrubicin can be combined, administered separately, or administered sequentially. Therefore, the invention also provides a pharmaceutical composition comprising picoplatin and amrubicin, and a pharmaceutically acceptable aqueous carrier, formulated for intravenous administration to a human.

The solutions can be physiological salt solutions that have been previously adjusted to be isotonic with suitable salts. In one embodiment of the invention, about 0.5 mg/ml of picoplatin is present in the aqueous infusion solution, and contains at least one pharmaceutically acceptable tonicity adjuster, such as NaCl, MgCl₂, CaCl₂, KCl and the like. Similar aqueous solutions of amrubicin can be employed. To achieve the preferred dosing, preferably about 200-300 mg of picoplatin is administered per dose, e.g., per intravenous infusion. Dosing of amrubicin can include about 40-60 mg of amrubicin per dose, e.g., per intravenous infusion or injection.

Over the course of treatment of the cancer, 2-10 doses of picoplatin can be administered, with 2-4 doses being typically administered, at intervals of about 21 days (three weeks). Intervals of up to six weeks, e.g., 3-4 weeks, can be employed if, for example, it is necessary to modify the treatment schedule to reduce side-effects. Over the course of treatment of the cancer, 2-10 treatments (of three doses over three consecutive days) of amrubicin can be administered, with 2-4 treatments being typically administered, at intervals of about 21 days (three weeks). Intervals of up to six weeks, e.g., 3-4 weeks, can be employed if, for example, it is necessary to modify the treatment schedule to reduce side-effects. As used above, the term “afflicted with lung cancer,” either 91-180 day progressive, resistant or refractory lung cancer, is also intended to encompass a patient who is afflicted with combined histology SCLC/non-small cell lung cancer. The picoplatin and amrubicin can be administered sequentially, in any order, or concurrently (simultaneously or overlapping).

In one embodiment of the present invention a patient afflicted with lung cancer, determined to have an absolute neutrophil count of at least 1.5×10⁹/L and a platelet count of at least 100×10⁹/L, a first dose of about 150 mg/m² picoplatin is administered. If the picoplatin is administered intravenously, it is preferably administered over 1-2 hours. A second dose of 150 mg/m² picoplatin is administered to said patient about 21 days after the first dose, and further dosing at this level is continued if hematological parameters remain stable. Amrubicin dosing can follow a similar schedule.

Best supportive care (BSC) for lung cancer comprises a number of palliative treatments that may also have limited therapeutic efficacy against lung cancer but are not considered to be curative. For example, in one embodiment of the invention, BSC includes one or more, and preferably all, of irradiation to control symptoms of metastatic cancer, administration of analgesics to control pain, management of constipation, and treatment of dyspnea and treatment of anemia, e.g., by transfusions, so as to maintain hemoglobin levels (i.e., ≧9 g/L). Other features of BSC for lung cancer are set forth below. In an embodiment according to the present invention, picoplatin and/or amrubicin can be administered in conjunction with a regimen of best supportive care. In another embodiment, the picoplatin and/or amrubicin can be the only chemotherapeutic anti-cancer agents administered to the patient. As lung cancer is predominantly a male disease, the patient can be a male patient.

The present method can further comprise administering an effective anti-emetic amount of a 5-HT₃ receptor antagonist and dexamethasone to the patient, for example, prior to step (c).

The present invention also provides method comprising administering a dosage form adapted for intravenous administration of picoplatin and amrubicin comprising: a solution comprising: (a) water; (b) a tonicity adjuster such as NaCl, in an amount effective to render the solution isotonic; (c) about 0.5 mg/mL dissolved picoplatin, and (d) about 0.5 mg/mL dissolved amrubicin, wherein administration of said dosage form is effective to treat resistant, refractory or progressive lung cancer. Separate unit dosage forms comprising an aqueous solution of picoplatin and an aqueous solution of amrubicin can be administered separately, sequentially, or concurrently (including simultaneously).

Additionally, the invention provides for the use of picoplatin alone to treat lung cancer, for example, NSCLC, specifically including refractory and 3rd line, and greater, levels of treatment. The administration of picoplatin can also be used to treat lung cancer, in conjunction with radiation treatment for any of the treatment groups described above. The lung cancer can be SCLC or NSCLC. These methods may also include the administration of amrubicin as described herein.

In various embodiments, the invention provides the use of an effective anti-cancer amount of picoplatin in conjunction with an effective anti-cancer amount of amrubicin for treating lung cancer in a human afflicted with lung cancer. The lung cancer can be small cell lung cancer (SCLC), or can be non-small cell lung cancer (NSCLC).

In various embodiments, the picoplatin and the amrubicin can be administered in one or more doses, wherein the human is optionally concurrently provided a regimen of best supportive care (BSC).

In various embodiments, the picoplatin can be in a dosage form adapted for administration by an oral route or an intravenous route, and the amrubicin is in a dosage form adapted for administration by an oral route or an intravenous route.

In various embodiments, the picoplatin can be administered once a day on day one of a two to four week treatment cycle, and at least two cycles of treatment are carried out.

In various embodiments, the amrubicin can be administered once a day for one to three days starting on day one of a two to four week treatment cycle, and at least two cycles of treatment are carried out.

In various embodiments, the treatment cycle can be a 21 day treatment cycle.

In various embodiments, a dose of about 5 mg/m² to about 150 mg/m² of picoplatin can be administered.

In various embodiments, a daily dose of about 5 mg/m² to about 45 mg/m² of amrubicin can be administered.

In various embodiments, the picoplatin, the amrubicin, or both, can be administered in an initial treatment dose, and then administered at about seven day intervals thereafter.

In various embodiments, the picoplatin can be administered daily for one day starting on day one of a 21 day treatment cycle and the amrubicin administered daily for the first three days of the 21 day treatment cycle.

In various embodiments, “treating” can comprise a treatment used as a first-line therapy wherein the lung cancer has not been previously treated with any other chemotherapeutic agents.

In various embodiments, “treating” can comprise a treatment used as a second-line or third-line therapy the patient is refractory, resistant, or relapsed/progressive within 91-180 days, after cessation of a first-line chemotherapy.

For example, the treatment can be first-line therapy for SCLC with extensive disease, alternatively can be first-line therapy for SCLC with limited disease and the treatment administered in conjunction with radiation therapy.

Or, the treatment can be second-line therapy for SCLC with extensive or limited disease that is refractory to initial chemotherapy or progressive within 6 months of completing first line, platinum-containing therapy.

Alternatively, the treatment can be first-line therapy for NSCLC with extensive disease, or can be first-line therapy for NSCLC with limited disease and the treatment administered in conjunction with radiation therapy.

Or, the treatment can be second-line therapy for NSCLC with extensive or limited disease that is refractory to initial chemotherapy or progressive within 6 months of completing first line, platinum containing therapy.

In various embodiments, the patient can have been previously been treated with radiation therapy.

Picoplatin or [SP-4-3]-ammine(dichloro)-(2-methylpyridine)platinum(II) (also known as NX 473, ZD0473, AMD 473, or [SP-4-3]-ammine(dichloro)-(2-methylpyridine)platinum(II)) is a new platinum agent that was developed to be effective against platinum-resistant (such as cisplatin-resistant) cell lines, and is intended for the treatment of solid tumors in humans (Raynaud, 1997; Holford, 1998 (both); Rogers, 2002). Like other platinum analogues, picoplatin causes cell death by the formation of covalent cross-links in DNA that interfere with DNA replication and transcription leading to cell death.

The name “picoplatin” has been designated as the United States Adopted Name (USAN), the British Approved Name (BAN) and the International Nonproprietary Name (INN) for this product. The molecular formula of picoplatin is C₆H₁₀N₂Cl₂Pt with a molecular weight of 376.14. The structural formula of picoplatin is:

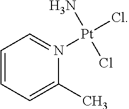

Picoplatin and processes for making picoplatin and for using picoplatin in treatment are disclosed and claimed in U.S. Pat. No. 5,665,771 (issued Sep. 9, 1997), and U.S. Pat. No. 6,518,428 (issued Feb. 11, 2003), and in PCT/GB0102060, filed May 10, 2001, published as WO2001/087313, which are incorporated herein by reference in their entireties.

In Phase I and II second-line studies with picoplatin, responses were seen in several tumor types, including ovarian, prostate cancer, and SCLC. Substantial nephro-, neuro- or ototoxicity have been observed with picoplatin only rarely in animal studies and in Phase I and Phase II trials (Beale, 2003; Treat; 2002; Giaccone, 2002; Gore, 2002). In Phase I studies of picoplatin, indications of activity were seen in subjects with ovarian cancer, NSCLC, SCLC, colorectal cancer, head and neck cancer, renal cell cancer, thymic cancer, pancreatic cancer, stomach cancer, leiomyosarcoma, liver cancer, mesothelioma, and prostate cancers (Beale, 2003).

Amrubicin, or (7S,9S)-9-Acetyl-9-amino-7-[(2-deoxy-β-D-erythro-pentapyranosyl)oxy]-7,8,9,10-tetrahydro-6,11-dihydroxy-5,12-nephthacenedione, is illustrated below as its hydrochloride salt.

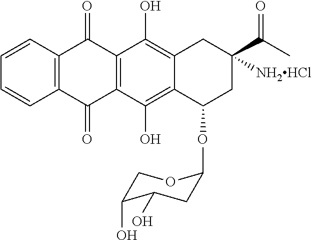

Amrubicin is a synthetic tetracycline derivative and can be prepared by various techniques, including those described in Japanese patent JP 3-5397 B. Amrubicin hydrochloride has been known to have several crystalline forms, a specific crystalline form of which is excellent in heat stability (JP 11-222497 A).

Amrubicin hydrochloride and the active metabolite amrubicinol show DNA intercalation activity, topoisomerase II inhibitory activity, DNA cleaving action mediated by stabilization of topoisomerase II cleavable complexes, and radical generation action. The primary mechanism of action is DNA cleaving action mediated by stabilization of topoisomerase II cleavable complexes.

Amrubicin, a synthetic 9-aminoanthracycline, is converted to an active metabolite, amrubicinol, through the reduction of its C-13 ketone group to a hydroxy group. Despite the similarity of its chemical structure to that of a representative anthracycline, doxorubicin, the mode of action of amrubicin differs from that of doxorubicin. Amrubicin and amrubicinol are inhibitors of DNA topoisomerase II, which exert cytotoxic effects by stabilizing a topoisomerase II-mediated cleavable complex, and are approximately 1/10 weaker than doxorubicin as a DNA intercalator. The in vitro cytotoxic activity of amrubicinol was 18 to 220 times more potent than that of its parent compound, amrubicin. In preclinical studies, amrubicin showed a more potent antitumor activity than doxorubicin in several human tumor xenografts implanted in nude mice, and caused almost no cardiotoxicity. The response rates to amrubicin at a dose of 45 mg/m² on days 1 to 3 in chemotherapy-naive patients with stage III or IV non-SCLC and extensive-stage SCLC were 25% and 79% on an intent-to-treat analysis, respectively. The major grade 3 or 4 toxicities were neutropenia (72.1%), leukopenia (52.5%), anemia (23.0%), thrombocytopenia (14.8%), anorexia (4.9%), and nausea/vomiting (4.9%) in a phase II trial.

### Treatment of Lung Cancer

The present invention provides a picoplatin dosage form that comprises a preferably sterile, preferably isotonic, aqueous solution adapted for intravenous (IV) administration. The solution, contains water, picoplatin at a concentration of about 0.3-0.75 mg/mL, e.g., about 0.75-1.0 wt. %, or about 0.5 mg/mL and a tonicity adjuster such as NaCl. In some embodiments, a preservative is not employed in the solution. The density of the solution can be about 1.005 g/mL.

TABLE 1A
Quantitative Composition Of Picoplatin Intravenous Infusion

| Ingredient | Function |
|---|---|
| Picoplatin | Active Ingredient (0.5 mg/ml) |
| Sodium Chloride USP | Tonicity Adjuster (0.9%) |
| Water for Injection USP | Solvent |

The present invention also provides an amrubicin dosage form that comprises a preferably sterile, preferably isotonic, aqueous solution adapted for intravenous (IV) administration. The solution, contains water, amrubicin at a concentration of about 0.3-0.75 mg/mL, e.g., about 0.75-1.0 wt. %, or about 0.5 mg/mL and a tonicity adjuster such as NaCl. In some embodiments, a preservative is not employed in the solution. The density of the solution can be about 1.005 g/mL. In some embodiments, the amrubicin dosage form can include additives such as lactose, L-cysteine HCl, and/or a pH regulator, such as hydrochloric acid and/or sodium hydroxide. The pH can be adjusted to about 2.4-3.0. The osmotic pressure ratio (to physiological saline) can be about 1.0-1.3 (dissolved in either saline or 5% glucose for injection).

TABLE 1B
Quantitative Composition Of Amrubicin Intravenous Infusion

| Ingredient | Function |
|---|---|
| Amrubicin | Active Ingredient (0.5 mg/mL) |
| Sodium Chloride USP | Tonicity Adjuster (0.9%) |
| Water for Injection USP | Solvent |

In one typical dosage form of amrubicin, 45 mg (titer)/m² (body surface area) of amrubicin is dissolved in approximately 20 mL of physiological saline or 5% glucose for injection. The dose can be administered intravenously once daily for a continuous 3-day period, after which a ‘drug holiday’ of about 3 weeks to about 4 weeks can be observed. The comprises “one course” or one treatment cycle, and the administration is then repeated. The dose can be decreased based on patient condition. Another suitable composition of amrubicin can formed according to the following table:

TABLE 1C
Amrubicin for Intravenous Infusion

| | For Injection 20 mg | | For Injection 50 mg | |
|---|---|---|---|---|
| Active Ingredient (per vial) | Amrubicin Hydrochloride | 20 mg (titer) | Amrubicin Hydrochloride | 50 mg (titer) |
| Additives (per vial) | Lactose | 50 mg | Lactose | 125 mg |
| | L-Cysteine HCl | 3.2 mg | L-Cysteine HCl | 8.0 mg |

| | |
|---|---|
| pH Regulator (HCl, NaOH); q.s. | pH Regulator (HCl, NaOH) ); q.s. |

| | |
|---|---|
| Color/Characteristics | Yellow-red powder or particles |
| pH* | 2.4-3.0 |
| Osmotic Pressure | 1.0-1.3 (Dissolved in Physiological Saline) |
| Ratio* (to physiological saline) | Approximately 1.3 (Dissolved in 5% Glucose for Injection) |

*5 mg (titer)/mL when the drug is dissolved in physiological saline or 5% glucose for injection.

The inventors herein have recognized that administration of picoplatin and amrubicin, for example intravenous administration, to the population of patients with lung cancer that is refractory or resistant to first-line organoplatinum therapy, or that progresses within the 91-180 day period after cessation of the first-line therapy, would be advantageous in terms of inhibiting further progression of the lung cancer and/or in prolongation of the patients' lives. Cancer that initially responds to first line therapy and then progresses within 90 days is referred to as resistant lung cancer. Cancer that initially responds to first line therapy, then progresses during the 91-180 day period can also be referred to as 91-180 day progressive lung cancer.

The picoplatin can be administered in doses ranging from about 60 mg/m² up to about 150 mg/m² per dose, which has been determined to be the maximum tolerated dose for second-line treatment of lung cancer, following initial platinum drug therapy. These dosage units refer to the quantity in milligrams per square meter of body surface area. The amrubicin can be administered in doses ranging from about 5 mg/m² up to about 45 mg/m² per dose

In another embodiment according to the invention, patients afflicted with lung cancer can be treated with picoplatin and amrubicin in conjunction with a regimen of best supportive care. The general guidelines used to provide subjects with best supportive care (BSC) are based on the NCCN Guidelines for lung cancer and for palliative care (NCCN Palliative Care Guidelines, 2007). In another embodiment, the picoplatin and amrubicin can be only chemotherapeutic anti-cancers agent administered to the patient selected for treatment.

The invention herein further includes a method of treating lung cancer wherein an effective anti-emetic amount of a 5-HT₃ receptor antagonist and dexamethasone are administered to the patient prior to administration of the picoplatin and amrubicin, in order to reduce the side effects of nausea and vomiting that can accompany administration of organoplatinum compounds. An example of a 5-HT₃ receptor antagonist that can be used according to the invention is ondansetron.

The administration of picoplatin and amrubicin can be carried out by any suitable technique known to those of skill in the art. For additional dosage forms that can be used to administer picoplatin and amrubicin are described in U.S. Provisional Patent Application Ser. Nos. 60/989,020 and 60/889,681, which are incorporated herein by reference. Suitable techniques for carrying out the combination therapy using picoplatin and amrubicin, including the use of radiation therapy can also be found in U.S. patent application Ser. No. 10/276,503, which is also incorporated herein by reference.

### Phase II Study

A Phase II study of picoplatin monotherapy for patients collectively afflicted with SCLC who have refractory, resistant or 91-180 day progressive disease, as defined herein, was carried out. A cohort of 77 patients, who had measurable disease, including 45 whose SCLC was unresponsive to first-line organoplatinum chemotherapy (cisplatin, carboplatin or oxaliplatin) (refractory) and 26 whose SCLC recurred within 90 days after cessation of first-line therapy (resistant), that is, 71 patients with refractory or resistant SCLC, plus 6 patients with 91-180 day progressive SCLC, were treated with picoplatin at a dosage of 150 mg/m² given intravenously over a period of 1-2 hours every 21 days. Picoplatin was provided as a sterile isotonic 0.5 mg/mL aqueous solution for IV infusion.

Patients received 1-10 cycles of picoplatin. A median number of dosage cycles of 2, and a mean number of dosage cycles of 3, were administered. Adverse events (AEs) were graded using the NCI CTCAE. The most frequently reported AEs of any severity are shown in Table 2, below. There was no grade 3 or 4 neurotoxicity, ototoxicity, or nephrotoxicity. There were no treatment-related deaths.

TABLE 2
Safety

| | All Grades | Grades 3/4 | Related to Drug |
|---|---|---|---|
| | (%) | (%) | (%) |
| Thrombocytopenia | 49 | 36 | 49 |
| Anemia | 46 | 17 | 42 |
| Neutropenia | 30 | 16 | 29 |
| Nausea | 27 | 1 | 22 |
| Dyspnea | 17 | 4 | 4 |
| Fatigue | 16 | 3 | 10 |
| Leukopenia | 16 | 3 | 16 |
| Constipation | 14 | 1 | 8 |
| Cough | 13 | 1 | 1 |
| Vomiting | 13 | 1 | 9 |
| Anorexia | 12 | 1 | 7 |
| Asthenia | 12 | 3 | 3 |

Tumor response was assessed every 6 weeks using RECIST criteria. Of 77 patients, three (4%) had partial response (PR), 34 (44%) had stable disease (unconfirmed PR+SD) and 36 (47%) had progressive disease. Disease control rate was 48% in the 77 patients. Median overall survival was 27 weeks (63 of 77 death events; 95% CI=21-32 weeks). The one-year survival rate was 18% (95% CI=11-28). Median progression-free survival was 9 weeks (71 of 77 progression events; 95% CI=7-12 weeks). Picoplatin monotherapy resulted in median survival that compares favorably with other reported therapeutic options for SCLC and had a reduced toxicity profile. The addition of amrubicin to the treatment regime can improve the treatment response and in some embodiments, lower the doses required for successful treatments.

### Dose Reduction

White blood cell counts (WBC) with differential and platelet counts and hemoglobin are obtained once between Days 11-15 of Cycles 1 and 2 to determine whether or not hematological toxicity has occurred. Subsequent doses for each subject are reduced by 30 mg/m² increments per cycle, up to two reductions, if toxicity is observed. Picoplatin is delayed up to 21 additional days and the dose reduced, if limits for absolute neutrophil count (ANC) and platelet counts are not met or for any other toxicity. Doses of picoplatin can be delayed in the event of unresolved hematological toxicities as described below. Doses of picoplatin are reduced in the event of hematological toxicity in the previous cycle, increased creatinine, or a change in body weight as described below. Once a subject has received a dose reduction, the dose may not be re-escalated. Subsequent treatments will continue at that level unless the toxicity recurs, in which case a further reduction of 30 mg/m² of the reduced dose may be made. Up to two dose reductions will be allowed. If an investigator determines that the degree of dose reduction should be greater than what is contained in these guidelines, investigator discretion shall take precedence to protect the safety of the subject. Similarly, if an investigator determines that a dose reduction should be applied earlier than suggested by these guidelines, investigator discretion shall take precedence to protect the safety of the subject. Similar dose reductions can be employed with respect to amrubicin when using the combination therapy.

The following hematological values should be obtained before picoplatin is administered: absolute neutrophil count (ANC) ≧1.5×109/L; and platelet count ≧100×109/L. If these criteria are not met, then laboratory tests should be measured at a minimum of weekly intervals to see if the required laboratory values are reached. In the event of an absolute neutrophil count less than 0.5×10⁹/L or a platelet count less than 25×10⁹/L, hematology values must be monitored at least twice a week until the neutrophil and platelet counts have improved to above these levels.

A maximum of 21 days is allowed for resolution of the events that do not meet the dosing criteria (i.e., to Day 42 of the cycle). Subjects who do not meet the re-dosing criteria by Day 42 (21 days post planned treatment) should be withdrawn from further treatment for reasons of toxicity.

A dose-reduction of 30 mg/m² is mandatory if any of the following criteria were observed during the previous cycle:

For hematological events: absolute neutrophil count (ANC) <0.5×10⁹/L for at least 5 days; or absolute neutrophil count <1.0×10⁹/L complicated with Grade ≧2 fever; or platelet count <25×10⁹/L; or not reaching a platelet count >100×10⁹/L and absolute neutrophil count >1.5×10⁹/L by Day 21. For non-hematological events (except nausea and vomiting or alopecia): treatment-related Grade 3 toxicity; or any Grade 4 toxicity.

For patients with abnormal serum creatinine, estimated creatinine clearance should be determined. If the calculated creatinine clearance is <60 mL/min, the subject should be monitored to ensure that there is no further deterioration in renal function. If a reduction in creatinine clearance is observed, the dose of picoplatin should be modified according to Table 3. Dose reductions are in the range of 30-60 mg/m² per administration.

TABLE 3

| Calculated creatinine | |
|---|---|
| clearance value | Dose modification |
| ≧60 mL/min | recommended dose |
| >40 to <60 mL/min | reduce by 30 mg/m² |
| >25 to ≦40 mL/min | reduce by 60 mg/m² * |
| ≦25 mL/min | discontinue treatment with picoplatin |

* If dose reduction would result in the patient receiving <90 mg/m² of picoplatin, the patient should be taken off study treatment.

A change in weight of 10% or more from that used in the previous calculation of body surface area requires a recalculation in body surface area and appropriate modification of drug dose.

### Phase III Study

A Phase III clinical study is carried out to demonstrate median survival superiority of picoplatin monotherapy with best supportive care (BSC) compared to best supportive care alone in patients with refractory or progressive disease within 180 days, including resistant and 91-181 day progressive, as defined above.

The plan is an open-label, randomized study of 21-day cycles of active study drug (picoplatin), continuing until progression plus BSC vs. BSC alone. Approximately 399 eligible subjects are randomly assigned to one of two treatment arms in a 2:1 ratio picoplatin plus BSC vs. BSC alone.

The dose of picoplatin is 150 mg/m² every 21 days. All subjects randomized to receive picoplatin receive 150 mg/m² of picoplatin on Day 1 of the first 21-day cycle administered over 1-2 hours. Picoplatin is provided as a sterile isotonic 0.5 mg/mL aqueous solution for IV infusion.

Subjects who are randomized to the picoplatin plus BSC arm receive anti-emetic therapy with a 5-HT₃ receptor antagonist and dexamethasone prior to administration of picoplatin and receive antiemetics following picoplatin administration, as necessary. All subjects receive BSC.

### Dose Reductions

White blood cell counts (WBC) with differential and platelet counts and hemoglobin are obtained once between Days 11-15 of Cycles 1, 2 and 3 to determine whether or not hematological toxicity has occurred. Subsequent doses for each subject may be reduced by 30 mg/m², increments per cycle if toxicity is observed. Picoplatin may be delayed up to 21 additional days and the dose may be reduced, if limits for absolute neutrophil count (ANC) and platelet counts are not met or for any other toxicity. Doses of picoplatin will be delayed in the event of unresolved toxicities as described below. A maximum of a 21-day delay is allowed for resolution of the events that do not meet the dosing criteria (i.e., to Day 42 of the cycle). Subjects who do not meet the re-dosing criteria by Day 42 (21 days post planned treatment) should be withdrawn from further treatment with study drug for reasons of toxicity, but should continue on study receiving BSC. Similar dose reductions can be employed with respect to amrubicin when using the combination therapy.

The dose of picoplatin will be reduced by 30 mg/m² decrements in the event of hematological toxicity in the previous cycle, decreased renal function, or significant non-hematological toxicity as described below. Once a subject has had a dose reduction, the dose must not be re-escalated. Subsequent treatments will continue at that reduced dose level unless the toxicity recurs, in which case a further reduction of 30 mg/m² of the reduced dose may be made. If an investigator determines that the degree of dose reduction should be greater than what is presented in these guidelines or that a dose reduction should be applied earlier than specified, investigator discretion shall take precedence.

The following hematological values must be obtained before picoplatin is administered: ANC ≧1.5×10⁹/L and platelet count ≧100×10⁹/L. If these criteria are not met, then laboratory tests should be repeated at a minimum of weekly intervals to see if the required laboratory values are reached. In the event of an absolute neutrophil count less than 0.5×10⁹/L or a platelet count less than 25×10⁹/L, hematology values must be monitored at least three times a week until the neutrophil and platelet counts have risen above these levels.

A maximum of a 21-day delay is allowed for resolution of toxicity (hematological or non-hematological) that does not meet the dosing criteria (i.e., to Day 42 of the cycle). Subjects who do not meet the re-dosing criteria by Day 42 (21 days post planned treatment) should be withdrawn from further picoplatin treatment for reasons of toxicity, but should continue receiving BSC on study.

A dose-reduction of 30 mg/m² is mandatory if any of the following criteria were observed during the previous cycle:

For hematological events: ANC <0.5×10⁹/L for at least 5 days; or ANC <1.0×10⁹/L complicated with Grade ≧2 fever (>39° C.); or Platelet count <25×10⁹/L; or Platelet count <100×10⁹/L and ANC <1.5×10⁹/L on Day 21. For non-hematological events (except alopecia): Treatment-related Grade 3 toxicity; or any Grade 4 toxicity; or Grade 3 or 4 nausea or vomiting while receiving recommended anti-emetic treatment.

If a reduction in creatinine clearance is observed, the dose of picoplatin should be modified according to Table 4.

TABLE 4

| Calculated Creatinine | |
|---|---|
| Clearance Value | Dose Modification |
| ≧50 mL/min | None |
| >35 to <50 mL/min | Reduce by 30 mg/m² |
| >25 to ≦30 mL/min | Reduce by 60 mg/m² |
| ≦25 mL/min | Discontinue treatment with picoplatin |

A change in weight of 10% or more from that used in the previous calculation of BSA requires a recalculation in BSA and appropriate modification of drug dose.

Picoplatin is preferably administered for six cycles or until either disease progression or unacceptable toxicity occurs. After discontinuation of picoplatin, all subjects continue to receive BSC and continue to be evaluated every three weeks until death or the end of the study.

The aim of this Phase III trial is to compare the efficacy and safety of picoplatin plus best supportive care (BSC) with BSC alone as second-line therapy for patients with SCLC who have disease that is refractory or progressive within 180 days (including 91-180 day progression) of completing first-line, platinum-containing chemotherapy. Toxicities, as in the Phase II study, are graded using the NCI CTCAE v3. The general guidelines are utilized to provide subjects with BSC and are based on the NCCN Guidelines for SCLC and for palliative care, as above.

The objectives of the study are to evaluate the following endpoints:

Overall survival. Patient survival, that is, prolongation of life, rather than the disease response rate, is the primary endpoint for measurement. Overall survival is measured from the date of randomization to the date of death from any cause. For each subject who is not known to have died, overall survival duration is censored at the date the patient was last known to be alive. It is believed that the increase in median overall survival will be statistically significant for patients treated with picoplatin compared to the survival for those treated with BSC alone, for example about 2-20 weeks longer, e.g., about 14 weeks.

The proportion of subjects who achieve an objective response (complete or partial response). Proportion of subjects with an objective response is measured as the proportion of subjects who achieve radiological evidence of a complete response (CR) or partial response (PR). For this analysis all subjects in the radiologically evaluable (RE) population, who do not meet the criteria as specified by RECIST for a CR or PR are included as if they did not have a response. The categorization of response uses the best overall response recorded from the initiation of study drug. Objective response requires a confirmatory exam documenting the response at least four weeks later.

The proportion of subjects who achieve disease control (complete or partial response, or stable disease). Proportion of subjects with disease control is measured as the proportion of subjects who achieve radiological evidence of a CR (complete response), PR (confirmed partial response), or SD (stable disease). For this analysis, all subjects in the RE population, who do not meet the criteria as specified by RECIST for a CR, PR, or SD are included as if they have progressed. Complete response and PR require a confirmatory exam documenting the response at least four weeks later. Stable disease is documented by a CT scan at least six weeks after the date of randomization, and this does not require a confirmatory exam.

All analyses of response or progression are based on the review of disease status by RECIST. These evaluations take place every six weeks.

### Phase IV Study

A Phase III clinical study is intended to be carried out to demonstrate median survival superiority of picoplatin and amrubicin combination therapy with best supportive care (BSC) compared to best supportive care alone in patients with refractory or progressive disease within 180 days, including resistant and 91-181 day progressive, as defined above. This study will combine the aspects of the picoplatin Phase I and Phase III studies described above with the addition of amrubicin in treatments.

### REFERENCES

All references below, and all other documents, patents, and publications referred to herein are incorporated by reference in their entireties.

Jemal A, Tiwari Ra, Murray T, et al. Cancer Statistics, 2004. CA Cancer J Clin 2004;54(1):8-29.

Vaporciyan A A, Kies M, Stevens C, et al. Cancer of the Lung; The Thorax. In: Cancer Medicine. 6 ed; 2003. p. 1385-1445.

Clark R, Ihde D C. Small-cell lung cancer: treatment progress and prospects. Oncology (Williston Park) 1998; 12(5):647-58; discussion 661-3.

Glisson B S. Recurrent small cell lung cancer: update. Semin Oncol 2003; 30(1):72-8.

Davies A, Evans W, Mackay J, et al. Treatment of recurrent small cell lung cancer. Hematology/Oncology Clinics of North America 2004; 18:387-416.

Murray N. Second-Line Chemotherapy for Small-Cell Lung Cancer. In: American Society of Clinical Oncology, Education Handbook; 2003. p. 667-71.

Jackman D M, Johnson B E. Small-cell lung cancer. Lancet 2005; 366(9494):1385-96.

Sundstrom S, Bremnes R M, Kaasa S, et al. Second-line chemotherapy in recurrent small cell lung cancer. Results from a crossover schedule after primary treatment with cisplatin and etoposide (EP-regimen) or cyclophosphamide, epirubicin, and vincristin (CEV-regimen). Lung Cancer 2005; 48(2):251-61.

National Comprehensive Cancer Network. Clinical Practice Guidelines in Oncology, Small Cell Lung Cancer, Principles of Supportive Care. 2006; Version 1:p. SCL-B.

Huisman C, Postmus P E, Giaccone G, et al. Second-line chemotherapy and its evaluation in small cell lung cancer. Cancer Treat Rev 1999; 25(4):199-206.

Giaccone G, Donadio M, Bonardi G, et al. Teniposide in the treatment of small-cell lung cancer: the influence of prior chemotherapy. J Clin Oncol 1988; 6(8):1264-70.

Raynaud F I, Boxall F E, Goddard P M, et al. cis-Amminedichloro(2-methylpyridine) platinum(II) (AMD473), a novel sterically hindered platinum complex: in vivo activity, toxicology, and pharmacokinetics in mice. Clin Cancer Res 1997; 3(11):2063-74.

Holford J, Raynaud F, Murrer B A, et al. Chemical, biochemical and pharmacological activity of the novel sterically hindered platinum co-ordination complex, cis-[amminedichloro(2-methylpyridine)] platinum(II) (AMD473). Anticancer Drug Des 1998; 13(1):1-18.

Holford J, Sharp S Y, Murrer B A, et al. In vitro circumvention of cisplatin resistance by the novel sterically hindered platinum complex AMD473. Br J Cancer 1998; 77(3):366-73.

Rogers P, Boxall F E, Allott C P, et al. Sequence-dependent synergism between the new generation platinum agent ZD0473 and paclitaxel in cisplatin-sensitive and -resistant human ovarian carcinoma cell lines. Eur J Cancer 2002; 38(12):1653-60.

Sharp S Y, O'Neill C F, Rogers P, et al. Retention of activity by the new generation platinum agent AMD0473 in four human tumour cell lines possessing acquired resistance to oxaliplatin. Eur J Cancer 2002; 38(17):2309-15.

Beale P, Judson I, O'Donnell A, Trigo J, Rees C, Raynaud F,. A Phase I clinical and pharmacological study of cis-diamminedichloro(2-methylpyridine) platinum II (AMD473). British Journal of Cancer 2003; 7(88):1128-1134.

Treat J, Schiller J, Quoix E, et al. ZD0473 treatment in lung cancer: an overview of the clinical trial results. European Journal of Cancer 2002; 38(8):S13-S18.

Giaccone G, O'Brien M E, Byrne M J, et al. Phase II trial of ZD0473 as second-line therapy in mesothelioma. Eur J Cancer 2002; 38 Suppl 8:S19-24.

Gore M E, Atkinson R J, Thomas H, et al. A Phase II trial of ZD0473 in platinum-pretreated ovarian cancer. Eur J Cancer 2002; 38(18):2416-20.

Ebi N, Kubota K, Nishiwaki Y, et al. Second-line chemotherapy for relapsed small cell lung cancer. Jpn J Clin Oncol 1997; 27(3):166-9.

Ardizzoni A, Hansen H, Dombernowsky P, et al. Topotecan, a new active drug in the second-line treatment of small-cell lung cancer: a Phase II study in patients with refractory and sensitive disease. The European Organization for Research and Treatment of Cancer Early Clinical Studies Group and New Drug Development Office, and the Lung Cancer Cooperative Group. J Clin Oncol 1997; 15(5):2090-6.

Gelmon, K. A., et al., A Phase 1 Study of AMD473 and Docetaxel Given Once Every Three Weeks In Patients with Advanced Refractory Cancer. Annals of Oncology 2004, 15, 1115-1124.

von Pawel J, Schiller J H, Shepherd F A, et al. Topotecan versus cyclophosphamide, doxorubicin, and vincristine for the treatment of recurrent small-cell lung cancer. J Clin Oncol 1999; 17(2):658-67.

Ardizzoni A. Topotecan in the treatment of recurrent small cell lung cancer: an update. Oncologist 2004; 9 Suppl 6:4-13.

Perez R P. Cellular and molecular determinants of cisplatin resistance. Eur J Cancer 1998; 34(10):1535-42.

Markman M. Topotecan as second-line therapy for ovarian cancer: dosage versus toxicity. Oncologist 2005; 10(9):695-7.

O'Brien MER, Ciuleanu T, Tsekov H, et al. in relapsed, resistant SCLC, oral topotecan plus supportive care offers survival benefit compared to supportive care alone. Proceedings from the 2005 Annual Chemotherapy Foundation Symposium 2005.

de Wet M, Falkson G, Rapoport B L. Small cell lung cancer: analysis of factors influencing the response to treatment and survival. Oncology 1994; 51(6):523-34.

Paesmans M, Sculier J P, Lecomte J, et al. Prognostic factors for patients with small cell lung carcinoma: analysis of a series of 763 patients included in 4 consecutive prospective trials with a minimum follow-up of 5 years. Cancer 2000; 89(3):523-33.

Bremnes R M, Sundstrom S, Aasebo U, et al. The value of prognostic factors in small cell lung cancer: results from a randomised multicenter study with minimum 5 year follow-up. Lung Cancer 2003; 39(3):303-13.

National Comprehensive Cancer Network. Small Cell Lung Cancer Guideline, Version 1 (2008). Available at: http://www.nccn.org/professionals/physician_gls/pdf/scic.pdf;

National Comprehensive Cancer Network. Palliative Care Guideline, Version 1 (2007). Available at: http://www.nccn.org/professionals/physician_gls/PDF/palliative.pdf.

J. Thorac. Oncol., 1 551 (2006).

Oncol. Rep., 15 837 (2006).

Jpn. J. Clin Oncol., 36 12 (2006).

Ann. Oncol., 16 430 (2005).

Osaki City Medical, 48 69 (2002).

Useful agents for administration with picoplatin and methods of treatment are also disclosed in include the platinum and non-platinum anticancer drugs disclosed in U.S. patent application Ser. No. 10/276,503, filed Sep. 4, 2003; Ser. No. 11/982,841, filed Nov. 5, 2007; Ser. No. 11/935,979, filed Nov. 6, 2007; Ser. No. 11/982,839, filed Nov. 5, 2007; in U.S. Pat. Nos. 7,060,808 and 4,673,668; in PCT WO/98/45331 and WO/96/40210.

The following patent applications are incorporated herein by reference in their entireties:

U.S. Ser. No. 61/027,387, filed Feb. 8, 2008, attorney docket no. 295.114pry

PCT Ser. No. ______, filed Feb. 6, 2009, attorney docket no. 295.114wol

U.S. Ser. No. 61/027,382, filed Feb. 8, 2008, attorney docket no. 295.115pry

PCT. Ser. No. ______, filed Feb. 6, 2009, attorney docket no. 295.115wol

U.S. Ser. No. 61/027,360, filed Feb. 8, 2008, attorney docket no. 295.116pry

U.S. Ser. No. 11/982,841, filed Nov. 5, 2007, attorney docket no. 295.093usl

U.S. Ser. No. ______, filed Feb. 6, 2009, attorney docket no. 295.131usl

1. A method for treating lung cancer in a human comprising administering to a human afflicted with lung cancer an effective anti-cancer amount of picoplatin and an effective anti-cancer amount of amrubicin.

2. The method of claim 1 wherein the lung cancer is small cell lung cancer (SCLC).

3. The method of claim 1 wherein the lung cancer is non-small cell lung cancer (NSCLC).

4-5. (canceled)

6. The method of claim 1 wherein the administration of the picoplatin is oral.

7. The method of claim 1 wherein the picoplatin is administered once a day on day one of a two to four week treatment cycle, and at least two cycles of treatment are carried out.

8. (canceled)

9. The method of claim 1 wherein the amrubicin is administered once a day for one to three days starting on day one of a two to four week treatment cycle, and at least two cycles of treatment are carried out.

10. The method of claim 9 wherein a daily dose of about 5 mg/m² to about 45 mg/m² of amrubicin is administered.

11. The method of claim 1 wherein the picoplatin, the amrubicin, or both, are administered in an initial treatment dose, and then administered at about seven day intervals thereafter.

12. The method of claim 7 wherein the treatment cycle is a 21 day treatment cycle.

13. The method of claim 12 wherein the picoplatin is administered daily for one day starting on day one of a 21 day treatment cycle and the amrubicin is administered daily for the first three days of the 21 day treatment cycle.

14. The method of claim 1 wherein the treatment is used as a first-line therapy wherein the lung cancer has not been previously treated with any other chemotherapeutic agents.

15. The method of claim 1 wherein the patient is refractory, resistant, or relapsed/progressive within 91-180 days, after cessation of first-line chemotherapy.

16. The method of claim 14 wherein the treatment is first-line therapy for SCLC with extensive disease.

17. The method of claim 14 wherein the treatment is first-line therapy for SCLC with limited disease and the treatment is administered in conjunction with radiation therapy.

18. The method of claim 14 wherein the treatment is second-line therapy for SCLC with extensive or limited disease that is refractory to initial chemotherapy or progressive within 6 months of completing first line, platinum-containing therapy.

19. The method of claim 14 wherein the treatment is first-line therapy for NSCLC with extensive disease.

20. The method of claim 14 wherein the treatment is first-line therapy for NSCLC with limited disease and the treatment is administered in conjunction with radiation therapy.

21. The method of claim 14 wherein the treatment is second-line therapy for NSCLC with extensive or limited disease that is refractory to initial chemotherapy or progressive within 6 months of completing first line, platinum containing therapy.

22. The method of claim 1 wherein the patient is first treated with radiation therapy.

23-78. (canceled)
